(19) United States
(12) Patent Application Publication
(10) Pub. No.: US 2004/0116802 A1
(43) Pub. Date: Jun. 17, 2004

(54) Medical imaging marker

(75) Inventors: Wayne G. Jessop, Simi Valley, CA (US); David W. Jessop, Simi Valley, CA (US)

Correspondence Address: THORPE NORTH & WESTERN, LLP, P.O. Box 1219, Sandy, UT, 84091-1219, US

(73) Assignee: Jessop Precision Products, Inc.

(21) Appl. No.: 10/680,556

(22) Filed: Oct. 6, 2003

Related U.S. Application Data
(60) Provisional application No. 60/416,092, filed on Oct. 5, 2002.

(51) Int. Cl. A61B 5/05

(52) U.S. Cl. 600/414; 600/426

(57) ABSTRACT

A medical imaging marker includes a marking body having a shape. The marking body can comprise a mixture of materials having different imaging properties. The particular properties of the different constituent materials of the mixture can be independently controlled. The relative amounts of the materials in the mixture can be varied. The mixture can be a conventional mixture, a suspension, a composite, a glass, or other mixture. The marker can be usable in a plurality of imaging techniques.

This application claims priority of U.S. Provisional Application No. 60/416,092, filed Oct. 5, 2002, the disclosure of which is hereby incorporated herein by reference.

### BACKGROUND OF THE INVENTION

1. Field of the Invention

The present invention relates generally to medical imaging markers. More particularly, the invention relates to medical imaging markers that can be adapted to a variety of medical imaging applications.

2. Related Art

Medical professionals utilize a variety of testing and diagnostic procedures to detect, analyze and treat medical conditions. Of the many tests available to the medical practitioner, radiological imaging is used extensively to create images of various anatomical structures within a patient's body. Radiological methods can include conventional X-Ray, Computerized Axial Tomography (“CAT”), Magnetic Resonance Imaging (“MRI”), Positron Emission Tomography (“PET”), and others.

These methods are used to create images of bone, tissue, vascular systems, tumors, etc., to aid medical professionals in locating disease and abnormalities that may otherwise be difficult to detect. The images created by such methods often contain only subtle differences in brightness and contrast that correspond to different anatomical features, structures, abnormalities, etc. For this reason, medical imaging markers are often placed on a patient's skin or embedded in a patient's body to serve as point of reference that can be identified in the final image. Such markers are useful in identifying relative positions of various structures, such as the distance from the skin surface to an object located below the skin surface. Markers are also used as informational indicators, such as designating anatomical orientation of the image, i.e., right or left, proximal or distal, toward a head or foot of a patient, etc. Markers are also used to identify particular bodily structure on or near the surface of the skin, such as nipples, moles, warts or other growths, scar tissues, etc. In this manner, a known or “normal” structure or object that appears in an image can be clearly identified and noted by a medical professional and not be misidentified or perceived as problematic when it is benign.

While such markers have proved at least partially effective, in many cases the markers interfere with the objective of the imaging technique. For example, markers formed of lead, mercury or steel often attenuate or scatter or block an x-ray beam, obscuring relevant structure, casting the underlying tissue into shadow, or otherwise producing unwanted artifacts on the image to be used by the medical professional. It has been noted that sometimes it is desirable to mark structure, but not completely obscure underlying structure by the virtue of the marker as would be the case using a traditional lead ball. Tissue detail within the “shadow” cast by such a marker is consequently not visible in the image formed.

Also, many conventional medical markers are limited to one particular type of medical imaging technique. That is, a particular marker may work well for conventional x-ray applications but perform poorly in MRI or CAT scans. For example lead or other metal balls can produce unwanted scatter in X-ray tomographic images, and are not generally useful or even usable in MRI imaging. Also, some sizes and shapes of markers may work better than others in particular applications. For instance, a large, spherical lead marker may work well for one application, but be nearly useless in another due to its relatively high degree of radiopacity. Also, some markers may produce relatively little “scatter” of imaging radiation in one type of imaging and/or magnitude of imaging radiation energy, but produce unreasonable amounts of scatter or other artifacts in another type of imaging or at another radiation level.

These problems, related to varying imaging techniques, and types, sizes and requirements of markers used therewith, can be further exacerbated in that manufacturers may produce markers only for one particular imaging technique. For example, one marker manufacturer may specialize in MRI markers, while another may concentrate on CAT scan markers. Thus, a professional who performs many types of medical imaging may have to stock a sizable array of markers from different manufacturers designed for different imaging techniques. This can lead to increased cost and difficulty in medical imaging.

In addition to the problems set forth above, health and environmental concerns are also limiting of the advantages of certain types of conventional medical markers. For example, many conventional markers are formed of lead, or contain lead, due to its radiation absorption advantages as used in a radiographic marker. Lead has long been used for, and in, markers due to its high degree of radiopacity and its relatively low cost. However, recognition, and mitigation of the negative health consequences of exposure to lead has led to increased costs in the production of lead markers, and in the disposal of the lead markers after use as well.

### SUMMARY OF THE INVENTION

It has been recognized that it would be advantageous to develop an environmentally safer, more versatile medical marking system that can be adapted for use in more than just one imaging technique. It has also been recognized that it would be advantageous to develop a system of markers that can provide a technician with a wide range of easily-identifiable markers for ease in selecting the optimal marker for use in a particular application. It has also been recognized that reducing potential harmful effects of lead used in markers can be beneficial.

The invention provides a marker that includes a mixture materials. The mixture can comprise a carrier and a marking material carried by the carrier. In a more detailed aspect, the marking material can be mixed in the carrier, and the mixture can be a true ionic mixture, other mixture, a composite, a suspension, a glass, or other combination of a plurality of materials having differing properties. In further detail, the marking material can be carried by a first carrier, for example comprising a solidified mixture such as a glass including silica and an additive providing increased visibility in a radiographic image, and formed into very small particles, e.g. spheres, of glass; the particles of this mixture themselves being carried in a second carrier, e.g. a viscous flowable material, a very viscous, very slow flowing material, a solidifying material such as a resin, or another material in which the particles are suspended or otherwise carried.

In a further more detailed aspect, the carrier and the marking material can have different radiation absorbing properties; and thus by variation of the amount of marking material carried in the carrier (or a composite of mixture particles carried in a second carrier), variation of the radiopacity, scatter, and other properties of the material of the marker can be varied. In another more detailed aspect, the properties of the marker can be controlled so as to make it usable in more than one kind of imaging technique.

In another more detailed aspect, the invention provides a medical imaging marker that can include a composite marking body that can have a predetermined shape. The marking body can comprise an at least partially radiopaque mixture of materials including at least two materials, one of which is selected from the group consisting of: silica; silicates; soda-lime glass; and leaded glass.

In a further more detailed aspect, an attachment substrate can also be provided, and the marking body can be disposed thereon. The attachment substrate can include means for removably attaching the marker to a patient's body.

In accordance with a more detailed aspect of the invention, a system of medical imaging markers is provided. Multiple marker types, each type having a marking body that is unique to the type, are provided. Each marking body type can have an operable characteristic that is different from another marking body type of the system. Thus markers having varying imaging properties, for example, different radiopaqueness, are provided. In further detail, such a system can include at least two medical imaging markers, each marker including an at least partially radiopaque marking body disposed upon an attachment substrate. Each marking body can exhibit a visually identifiable color that is different from a visually identifiable color exhibited by another marking body.

In accordance with a more detailed aspect of the present invention, a medical imaging marker is provided and includes a composite marking body including a viscous carrier capable of application to a patient's body in a variety of patterns and application sizes. A multiplicity of at least partially radiopaque particles can be disposed within and carried by the viscous carrier. The radiopaque particles can provide an at least partially radiopaque characteristic to the marking body. In further detail, the radiopacity can be varied by variation of the concentration of the particles in the carrier. The viscous carrier can be a material imageable in MRI, whereby the marker can be used in both MRI and X-ray imaging, and other imaging methods.

In accordance with a further more detailed aspect of the present invention, a medical imaging marker is provided and includes a marking body that can exhibit a visibly identifiable color which is associated with an operable characteristic of the marker. An attachment substrate can also be provided, on which the marking body can be disposed. The attachment substrate can include means for removably attaching the marker to a patient's body.

In accordance with a further more detailed aspect of the present invention, a medical imaging marker is provided and includes a carrier material capable of being formed in a predetermined shape. A mixture constituent marker material can disposed within and carried by the carrier material, the constituent marker material having an operable characteristic associated therewith. An attachment substrate can also be provided, on which the carrier material can be disposed. The attachment substrate can include means for removably attaching the marker to a patient's body. Color of the marker material, carrier, or substrate or combinations thereof, can be used to visually indicate the operable characteristic.

Additional features and advantages of the invention will be apparent from the detailed description which follows, taken in conjunction with the accompanying drawings, which together illustrate, by way of example, features of the invention.

### BRIEF DESCRIPTION OF THE DRAWINGS

FIG. 1 is a perspective view of a medical imaging marker in accordance with an embodiment of the present invention, including a composite marking body attached to a substrate, an optional tab feature of the substrate being shown in outline;

FIG. 1A is a schematic representation of various colors of a composite marking body usable in connection with the marker of FIG. 1;

FIG. 2 is a perspective view of a system of medical imaging markers in accordance with an embodiment of the invention, shown mounted on a backing tape for dispensing in one embodiment and a backing sheet in outline in another embodiment;

FIG. 3 is a perspective schematic representation, partially in cut-away, of an example composite marking body in one example embodiment.

FIG. 4, is a perspective schematic representation, partially in cut-away, of an example composite marking body having selected shape characteristics, illustrating principles of the invention;

FIG. 5A is a perspective view of another medical imaging marker in accordance with an embodiment of the invention;

FIG. 5B is a perspective view of another medical imaging marker in accordance with an embodiment of the invention;

FIG. 5C is a perspective view of another medical imaging marker in accordance with an embodiment of the invention;

FIG. 6 is a perspective view of a medical imaging marker in accordance with another embodiment of the invention; and

FIG. 7 is a perspective view of a medical imaging marker in accordance with another embodiment of the invention.

### DETAILED DESCRIPTION

Reference will now be made to the example embodiments illustrated in the drawings, and specific language will be used herein to describe the same. It will nevertheless be understood that no limitation of the scope of the invention is thereby intended.

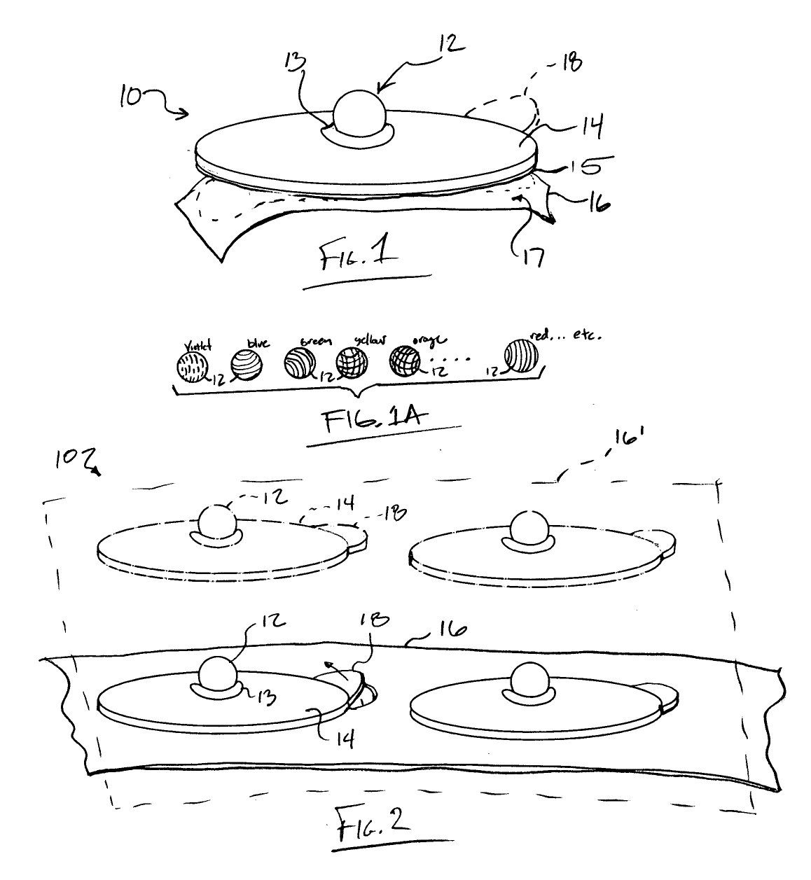

With reference to FIG. 1, an example medical imaging marker 10 is provided in accordance with one aspect of the invention. The marker can include a marking body 12 which can comprise an at least partially radiopaque material. As used herein, the term “radiopaque” is to be understood to mean a material or property which absorbs, blocks, attenuates or interferes with imaging radiation, so as to enable an image artifact to be created in the imaging process corresponding in a predictable way with the location of the marker. The marking body 12 can be disposed upon an attachment substrate 14 that can be configured to be removably attached to a patient's body. An adhesive 13, for example a polymer adhesive, can adhere the marking body to the substrate. An adhesive layer 15 can be provided to make the marker attachable to a patient. In one aspect of the invention, a backing layer 16 is provided, which can include a surface including a release 17, to enable the attachment substrate to function as a peel-off label as is known in the art. The attachment substrate and adhesive can be removed from the backing to expose the adhesive layer 15, which can then be placed on a patient to attach the substrate (and thus the marker) to the patient's skin.

In one embodiment, a lift tab 18 can be provided to make removal of the marker from the backing 16, and subsequently from the patient's skin (not shown), more convenient. The adhesive 15 under the lift tab can be rendered ineffective, for example by undercutting the backing, and/or deadening the adhesive locally, so that the lift tab can be more easily grasped and thereby the attachment substrate can be more easily removed from the backing and afterward from the patient.

The imaging body 12 in the illustrated embodiment comprises a mixture of materials. It can be, for example, a glass such as soda lime glass, leaded glass, or another glass mixture. The glass can include a colorant or be left relatively colorless. It has been found that certain glass mixtures exhibit the quality of being partially radiopaque, thus allowing structure underneath to be less obscured in conventional X-ray and CT scanning than would be the case using a conventional lead ball, for example. Also, it has been observed that such marking bodies exhibit less scattering of imaging radiation, and accordingly produce less undesirable image artifacts caused by such scattering.

The radiopacity of the marking body 12 can be adjusted. Different glass mixtures having different proportionate amounts of constituent materials exhibit different radiopacities. Additional additives, such as coloring agents, suspended particulates, as well as air bubbles or other entrained materials, when mixed into the glass mixture, can further change its radiopacity.

Further, color, as a visual indicia, can be used to indicate different operative characteristics of the markers. For example with reference to FIG. 1A, different colors of the marking body can be provided. The different colors can correspond to different radiopacities of the marking body for example, or to different materials of construction (and accordingly to different applications to which the marker is appropriate (e.g. CAT, MRI, PET, etc.) to name another example. The colorant additive can itself alter the relevant property, for example radiopacity, and so this should be taken into account. But the resulting advantage is that markers of the same size can have different radiopacity, and the color, rather than size of the marker body, can be used to indicate the relative radiopacity of the marker (10 in FIG. 1) in one example. In another example one color may indicate that the marker is appropriate for a particular use, e.g. MRI, while another indicates that the marker is appropriate for another, such as CT scan applications.

With reference again to FIG. 1, the marker body 12 and the adhesive 13 can be a single dollop of the same material mixture. For example, the mixture can be a composite, suspension, or other mixture of materials, the salient properties of which is that it will cure into a substantially solid marker body adhering to the substrate 14, and that it has a marking property. That is to say, in the later case, that for the intended use it will provide a mark on the resulting image in accordance with the imaging technique used. This will be discussed in further detail below.

With reference to FIG. 2, in one embodiment markers 10 can be dispensable from a backing 16 configured as a tape. In the illustrated embodiment the lift tab 18 is undercut so that the backing lifts with the tab. In another embodiment the markers can be dispensed from a backing configured as a sheet 16′ carrying a multiplicity of markers individually removable. The particulars of providing sheet and tape dispensable markers having peel-off adhesive backed substrates 14 are well known, and thus are not set out in detail here.

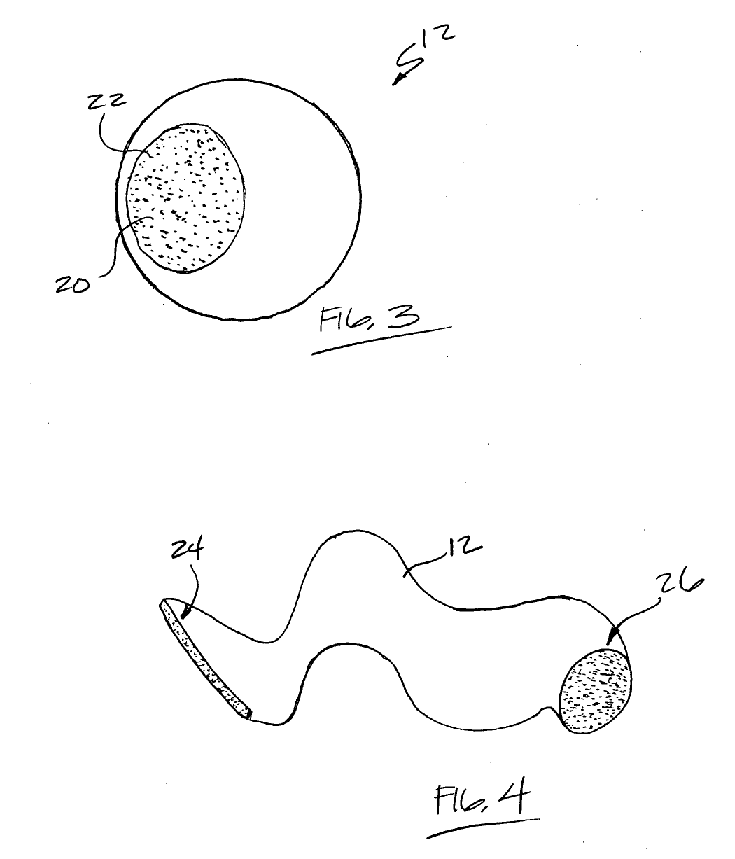

With reference now to FIG. 3, in the illustrated embodiment a marking body 12 comprises a mixture of a carrier 20 and a marker material 22. By mixture what is meant herein is a combination of the materials, whether a suspension, a composite, a liquid (including amorphous solids such as glasses), or other combination, which materials integrally work together to provide the desired properties of the marking body. Note that in one embodiment the marker material can be small glass particles, e.g. microspheres, which are themselves a mixture of materials. In other words, the marking material can be a mixture within a mixture. For example, soda-lime glass microspheres can be used as the marker material 22, carried by an adhesive material serving as the carrier material 20. Other carrier materials and other marker materials can be used.

In another example, metals such as lead, bismuth, etc, high polymers, or another material which attenuates imaging radiation well, can be provided in the form of small particulates 22 suspended in a carrier material 20. The carrier envelopes and encapsulates the marker material, mitigating exposure hazard, if any. Moreover the carrier can be selected to be moldable, malleable, flowable, solidifiable, hard, soft, elastomeric, as desired. As illustrated in FIG. 4, the marking body can be made into various shapes, from thin (e.g. at 24) to bulbous (e.g. at 26), wire-like, spherical (as in FIG. 3), square, torroidal, etc. Greater flexibility in properties of the marking material is possible at less cost, due at least in part to the fact that certain properties can be varied by variation of the marking material, e.g. by varying the material itself, or its proportion in the mixture. Whereas substantially independently the properties of the carrier can be varied to make it more or less viscous, flowable, hard, soft, shaped or unshaped, colored, textured, adhesive, non-adhesive, etc.

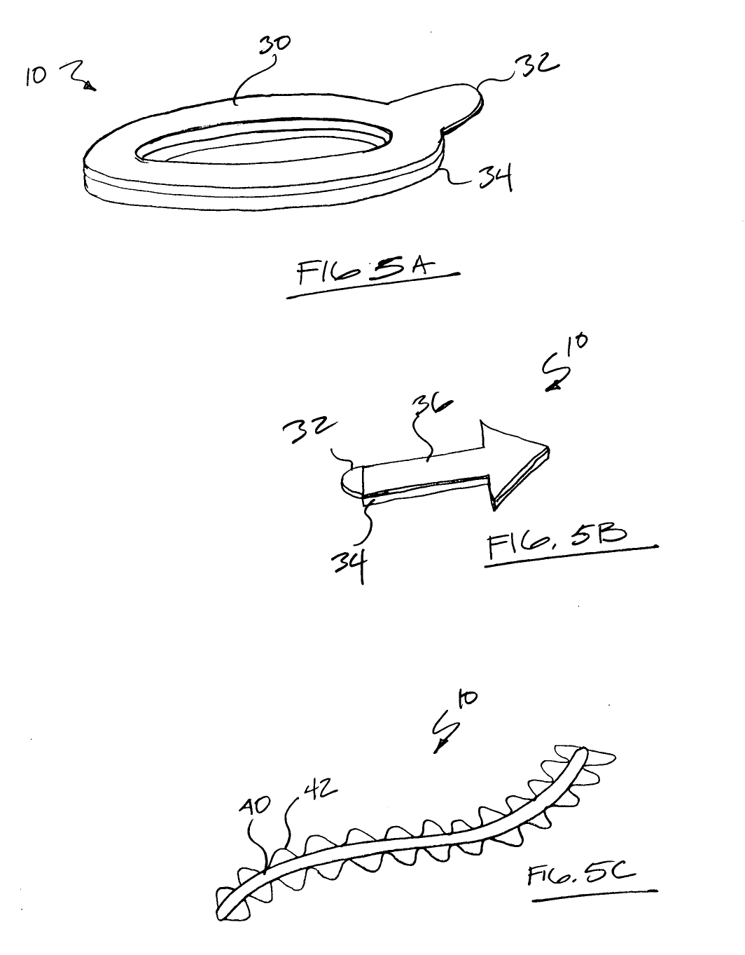

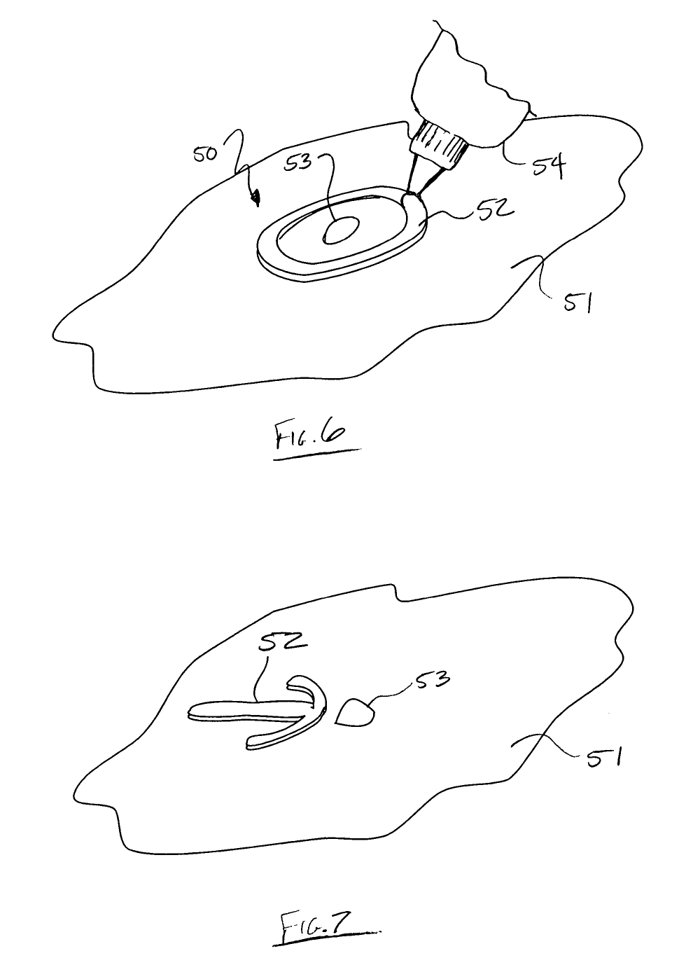

As an example of this variability, with reference to FIG. 5A, in one example the marker 10 can comprise an O-shaped substrate 30, which can have a lift tab 32, and has an adhesive mixture 34 which includes an adhesive material as the carrier and a marker material mixed therein (e.g. in suspension, forming a composite, for example). The circular shaped adhesive layer can have a radiopacity appropriate to be discernable to surroundingly mark a structure such as a growth, nipple, mole, etc. while not completely obscuring underlying structure in an x-ray image for example.

As another example, with reference to FIG. 5B, the marker can be similarly configured, having an adhesive carrier and marking material mixture layer 34, lift tab 32, but a substrate 36 shaped as an arrow. The Marking material, being also in the shape of an arrow, can produce an arrow-shaped indication on a radiographic image.

In a further example, and with reference now to FIG. 5C, a carrier material and marker material mixture can be configured as a formable wire-like marking body structure 40 disposed on an adhesive substrate 42 configured to allow the wire-like structure to be bendable. For example a curable polymeric adhesive material that is malleable when cured can be used as a carrier material and soda-lime glass microspheres as the marker material to form the mixture forming the marking body.

With reference to FIGS. 6 and 7, in another embodiment a marker 50 can be supplied in a viscous flowable form, which adheres to a patient's skin 51 to mark a structure, e.g. a growth 53. The carrier/marker material mixture 52 is flowable initially, and in one embodiment can be of relatively high viscosity and simply remain on the skin in the form dispensed from a dispenser 54 until wiped/washed off. In another embodiment it can comprise a light or air-curing material which stiffens after being dispensed to allow contact with equipment without distortion or rubbing off. As will be appreciated, the thickness, shape and location of the marker 50 are all controlled by the medical professional using the marker on a particular patient.

In one embodiment the viscous carrier can be a material imageable using an MRI technique. In such an example, the marker can be used in both MRI and with radiation exposure based radiological techniques to provide an indication of location, direction, etc.

With all the above examples, color of the carrier or the marking material (e.g. if the carrier is clear) or of another additive in the mixture can indicate some operative characteristic of the marker 10, 50, etc. This can be an indication of some parameter of the material such as radiopacity, or some indication of applicability to a particular imaging technique, such as one color for X-ray-, one for X-ray tomographic-, one for PET- one for MRI-, type imaging techniques, to name some examples.

Returning to FIG. 3, in further detail, in one embodiment the marking body 12 can comprise a sphere, adapted for use in tomography, MRI and other imaging techniques where a plurality of images taken along different directions (or through different planes) will be used. Moreover, such marking bodies, particularly those formed of non-metallic mixtures of carrier 20 and imaging material 22 can be used in more than one imaging technology, for example MRI and PET scans, in addition to those imaging techniques mentioned using X-ray radiation. For example, an encapsulated liquid carrier with glass beads can be used in MRI and CAT, for example, if the carrier is a liquid imageable in MRI, and the beads are imageable in CAT, to name one example.

Particularly with respect to the glass ball embodiments mentioned, where the carrier is, for example conventional silica, silicates, etc. having a first radiopacity and the imaging material is an additive such as the lime, soda ash, Na/Ca or other oxides, e.g., Na₂O, CaO, Al₂O₃, B₂O₃, MgO, PbO, K₂O, TiO₂, LiO₂, etc. conventionally used, having a second radiopacity, the radiopacity of the resulting ionic mixture can be controlled by controlling the relative amounts of the imaging material to the carrier material. Moreover, admixtures of lead and other metals, polymeric materials, etc. where the imaging material is present as a separate phase, are also usable to control an operative property, such as radiopacity. Such a material in the latter case is more of a suspension or composite than a true “mixture;” but, per the above, such amalgamations of materials are intended to be within the definition of the word “mixture” whether or not boundaries between discrete materials in the mixture are, or are not, present.

Note also, that the carrier material in the above examples is the predominant “imaging” material in the MRI application embodiments. This should indicate to the reader that the particular word choices are for convenience of exposition and the necessity of setting forth the claims in a verbal language, but that language does not always precisely fit the novel concepts presented. A point to bear in mind is that one or more salient characteristics of the respective materials of the mixture are different with respect to how they interact with one or more imaging techniques to produce a mark on the resulting image. That concept is more telling than the particular terminology applied thereto to try to approximate the inventive subject matter in a particular expression in a written language.

It is to be understood that the above set forth descriptions and arrangements are illustrative of the application for the principles of the present invention. It will be apparent to those of ordinary skill in the art that numerous modifications can be made without departing from the principles and concepts of the invention as set forth in the claims. Additional applications of the principles of the inventions as illustrated herein, which would occur to one skilled in the relevant art and having possession of this disclosure, are to be considered within the scope of the invention.

What is claimed is:

1. A medical imaging marker, comprising:
a marking body, having a predetermined shape, the marking body including an at least partially radiopaque material selected from the group consisting of: silica; silicates; soda-lime glass; and leaded glass; and
an attachment substrate, on which the marking body is disposed, the attachment substrate including means for removably attaching the marker to a patient's body.

2. The marker of claim 1, wherein the marking body is formed of a nonmetallic material.

3. The marker of claim 1, wherein the marking body is formed of a nonleaded material.

4. The marker of claim 1, wherein the predetermined shape is a substantially spherical shape.

5. The marker of claim 1, wherein the marking body exhibits a visibly identifiable color which is associated with an operable characteristic of the marker.

6. The marker of claim 5, wherein the operable characteristic of the marker includes a characteristic selected from the group consisting of: a size of the marker; a radiopacity of the marker; a radiolucency of the marker; a type of medical imaging with which the marker will be used; and a biological structure which is to be marked by the marker.

7. A system of medical imaging markers having varying radiopaqueness, comprising:
at least two medical imaging markers, each marker including an at least partially radiopaque marking body disposed upon an attachment substrate;
each marking body exhibiting a visually identifiable color that is different from a visually identifiable color exhibited by another marking body; and
each marking body having an operable characteristic that is different from another marking body of the system.

8. The system of claim 7, wherein the marking bodies of each imaging marker have substantially the same size and shape.

9. The system of claim 7, wherein the visually identifiable color exhibited by each marking body is one of a: primary; secondary; or tertiary color.

10. The system of claim 7, wherein the visibly identifiable color is associated with the operable characteristic of the marker.

11. The system of claim 7, wherein the operable characteristic of the marker includes a characteristic selected from the group consisting of: a size of the marking body; a radiopacity of the marker; a radiolucency of the marker; a type of medical imaging with which the marker will be used; and a biological structure which is to be marked by the marker.

12. A medical imaging marker, comprising:
a viscous carrier capable of application to a patient's body in a variety of patterns and application sizes; and
a multiplicity of at least partially radiopaque particles disposed within and carried by the viscous carrier, the radiopaque particles providing an at least partially radiopaque characteristic to the viscous carrier.

13. The marker of claim 12, wherein the viscous carrier includes an adhesive material.

14. The marker of claim 12, wherein the viscous carrier is curable by contact with an atmospheric environment to facilitate curing of the viscous carrier into a semi-solid state.

15. The marker of claim 12, wherein the marking material is disposed upon a substrate, and the substrate is attachable to a patient's body.

16. The marker of claim 12, wherein the marking material is disposed between at least two substrates, one of the substrates being removable to facilitate application of the marking material to a patient's body, and the remaining substrate being configured to remain attached to the marking material to provide a protective cover over the marking material while applied to the patient's body.

17. A medical imaging marker, comprising:
a marking body, exhibiting a visibly identifiable color which is associated with an operable characteristic of the marker; and
an attachment substrate, on which the marking body is disposed, the attachment substrate including means for removably attaching the marker to a patient's body.

18. The marker of claim 17, wherein the operable characteristic of the marker includes a characteristic selected from the group consisting of: a size of the marking body; a radiopacity of the marker; a radiolucency of the marker; a type of medical imaging with which the marker will be used; and a biological structure which is to be marked by the marker.

19. The system of claim 17, wherein the visually identifiable color exhibited by each marking body is one of a: primary, secondary or tertiary color.

20. A medical imaging marker, comprising:
a carrier material capable of being formed in a predetermined shape;
a constituent material disposed within and carried by the carrier material, the constituent material having an operable characteristic associated therewith; and
an attachment substrate, on which the carrier material is disposed, the attachment substrate including means for removably attaching the marker to a patient's body.

21. The marker of claim 20, wherein the constituent material is selected from the group consisting of: silica; sodium oxide; calcium oxide; lead oxide; aluminum oxide; boric oxide; soda; and potash.

22. The marker of claim 20, wherein the predetermined shape is a substantially spherical shape.

23. The marker of claim 20, wherein the carrier material and constituent material cooperatively exhibit a visibly identifiable color which is associated with the operable characteristic of the marker.

24. The marker of claim 23, wherein the operable characteristic of the marker includes a characteristic selected from the group consisting of: a size of the marker; a radiopacity of the marker; a radiolucency of the marker; a type of medical imaging with which the marker will be used; and a biological structure which is to be marked by the marker.
